(19) United States
(12) Patent Application Publication
(10) Pub. No.: US 2022/0087926 A1
(43) Pub. Date: Mar. 24, 2022

(54) COMPOUND COMPOSITION WITH ANTI-OXIDATION AND ANTI-AGING EFFECTS AND PREPARATION METHOD AND APPLICATION THEREOF

(71) Applicants: Chenland Nutritionals Inc.; Qingdao Chenland Pharmaceutical Technology Development Co., Ltd., QINGDAO (CN)

(72) Inventors: Shengcan Zou, QINGDAO (CN); Zengliang Zhang, QINGDAO (CN); Min Wang, QINGDAO (CN); Sai Lu, QINGDAO (CN); Zhuqing Liu, QINGDAO (CN); Jiancheng Zong, QINGDAO (CN); Shanglong Wang, QINGDAO (CN)

(21) Appl. No.: 17/207,740

(22) Filed: Mar. 22, 2021

Related U.S. Application Data
(63) Continuation of application No. PCT/CN2020/120583, filed on Oct. 13, 2020.

(30) Foreign Application Priority Data
Sep. 24, 2020 (CN) 202011016232.8

(51) Int. Cl. A61K 8/9794; A61K 8/9789; A61K 8/9728; A61Q 19/08; A23L 33/105; A61K 36/076; A61K 36/8965; A61K 36/258; A61K 36/64; A61K 36/738; A61K 36/537; A61K 9/48; A23L 33/00

(52) U.S. Cl. CPC A61K 8/9794; A61K 2800/522; A61K 8/9728; A61Q 19/08; A23L 33/105; A61K 36/076; A61K 36/8965; A61K 36/258; A61K 36/64; A61K 36/738; A61K 36/537; A61K 9/48; A23L 33/30; A61K 2800/92; A61K 8/9789

(57) ABSTRACT

A compound composition with anti-oxidation and anti-aging effects and preparation method and application thereof we disclosed. 5-8 kinds of traditional Chinese medicine extracts from separate extraction of Panax ginseng C. A. Meyer, Poria, Rehmannia glutinosa, Cistanche deserticola Ma, Salviae Miltiorrhizae, Codonopsis Radix, Persicae Semen and Ophiopogon japonicus (Linn.f.) Ker-Gawl. are compounded with chitooligosaccharide to prepare the compound composition with anti-oxidation and anti-aging effects. The compound composition has no toxic side effects, is easy to be absorbed, and can be used for delaying aging, repairing DNA damage and anti-oxidation damage, and can also reduce the risk of geriatric diseases. Moreover, the compound composition can be used as a raw material of dietary supplement or health food to improve telomerase content and delay aging. Therefore, the compound composition is suitable for promotion and application.

### CROSS-REFERENCE TO RELATED APPLICATIONS

This application is a continuation of International Patent Application No. PCT/CN2020/120583 with a filing date of Oct. 13, 2020, designating the United States, now pending, and further claims priority to Chinese Patent Application No. 202011016232.8 with a filing date of Sep. 24, 2020. The content of the aforementioned applications, including any intervening amendments thereto, we incorporated herein by reference.

### TECHNICAL FIELD

The present disclosure relates to natural medicines, and more specifically, to a compound composition with anti-oxidation and anti-aging effects, preparation and application thereof.

### BACKGROUND

Traditional Chinese medicine (TCM) holds that aging is caused by various reasons, such as kidney deficiency, spleen deficiency, blood stasis and phlegm turbidity. And invigorating the kidney, invigorating the spleen, activating blood circulation and removing blood stasis, and removing phlegm can all delay aging. Among them, invigorating the kidney and spleen is the basic way to delay aging, activating blood circulation and removing phlegm is the important way to delay aging. However, deficiency and excess mixed with many factors lead to aging more. “Deficiency” is mainly concentrated in spleen and kidney deficiency, and “excess” is concentrated in two categories: phlegm and blood stasis.

In addition, modern medical research on the causes of aging includes: aging free radical theory, telomere and telomerase theory, etc. However, the increase in the level of free radicals or the absence of antioxidants leads to the decrease of the anti-oxidation capacity and the increase of cytotoxicity of the organism, and finally causes irreversible change and damage of the biofilm, the amino acid chain and the molecular structure of DNA, which leads to the generation of aging related degenerative diseases and accelerates the aging process. Telomere, as the substrate of telomerase, ensures the integrity of chromosome replication and determines the cell cycle. Moreover, telomere length is related to cell division times and life limit. Scientists expressed telomerase in aging cells, which not only reversed cell aging, but also began to use glucose to synthesize nucleotides. The regulation of aging depends on the regulation of telomere by telomerase and the joint operation of telomere and telomerase.

Health food or food with anti-aging function at present mostly take anti-oxidation and immunity enhancement as the breakthrough point, mainly using grape seed extract, resvertrol, astaxanthin and other strong antioxidants, and American ginseng, Lycium barbarum and other traditional Chinese medicine to enhance immunity. At the same time, cell aging, genes and other deep-seated aging have also gradually entered the public's field of vision. In view of the complexity and uncertainty of aging mechanism itself, the phenomenon of aging can't be explained by a single mechanism, so it is necessary to carry out a multi-faceted and multi-level in-depth exploration. Similarly, delaying aging also needs to start with multi-component, multi-level and multi-action mechanism.

Based on the above, at present, most of the existing anti-aging products on the market start from scavenging free radicals, and the existing free radical scavengers are mainly single extracts or vitamin products, such as grape seed extract, resveratrol, astaxanthin, vitamin E, etc., which have very limited ability to scavenge free radicals. And the range of free radicals that can be scavenged is narrow, and only a part of free radicals can be effectively scavenged. Therefore, it is difficult to truly compensate for the decline in free radical scavenging capacity due to aging, and no in-depth study has been conducted on specific aging signs, contents of aging-related enzymes and changes in regulatory factors in human body.

Therefore, it is an urgent problem for those skilled in the art to develop a compound composition which can promote healthy cells to delay aging and resist oxidation, and has no toxic side effects, is easy to be absorbed, and ha anti-oxidation and anti-aging effects.

### SUMMARY

In view of the above, an object of the present disclosure is to provide a compound composition with anti-oxidation and anti-aging effects. The compound composition has no toxic side effects and is easy to be absorbed. It can be used as a raw material of dietary supplement or health food to delay aging, repair DNA damage and anti-oxidation damage, and reduce the risk of geriatric diseases.

In order to achieve the above object, technical solutions of the present disclosure are specifically described as follows.

In the present disclosure, through the organic combination of traditional Chinese medicine compound modified Qiongyu ointment and modern marine component chitooligosaccharide, aging is effectively delayed by both traditional Chinese medicine and modern science. The selected classical ancient prescription Qiongyu ointment, which has the effect of nourishing Yin, moistening lung, invigorating spleen and strengthening lung, and the addition of Cistanche deserticola Ma and Salviae Miltiorrhizae can play the role of delaying aging in many aspects. And the disclosure develops a new way for the application of Qiongyu ointment.

In a first aspect, the disclosure provides a compound composition with anti-oxidation and anti-aging effects, including traditional Chinese medicine extracts mixed with chitooligosaccharides in a weight ratio of (3˜8): 1. The traditional Chinese medicine extracts are prepared by extracting the following Chinese medicinal materials respectively and compounding:

7%-15% by weight of Panax ginseng C. A. Meyer;

14%˜35% by weight of Poria;

14%˜35% by weight of Rehmanniae glutinosa;

14%˜35% by weight of Cistanche deserticola Ma;

7%˜15% by weight of Salviae Miltiorrhizae;

0%˜10% by weight of Codonopsis Radix;

0%˜10% by weight of Persicae Semen; and

0%˜10% by weight of Ophiopogon japonicus (Linn.f.) Ker-Gawl.

Preferably, the traditional Chinese medicine extracts are prepared by extracting the following Chinese medicinal materials respectively and compounding:
12%-15% by weight of Panax ginseng C. A. Meyer;
25%˜30% by weight of Poria;
20%˜25% by weight of Rehmanniae glutinosa;
20%˜25% by weight of Cistanche deserticola Ma;
12%˜15% by weight of Salviae Miltiorrhizae,
2%˜3% by weight of Codonopsis Radix;
1%˜2% by weight of Persicae Semen; and
1%˜2% by weight of Ophiopogon japonicus (Linn.f.) Ker-Gawl.

Preferably, the traditional Chinese medicine extracts are prepared by extracting the following Chinese medicinal materials respectively and compounding:
13% by weight of Panax ginseng C. A. Meyer;
26% by weight of Poria;
22% by weight of Rehmanniae glutinosa;
22% by weight of Cistanche deserticola Ma;
13% by weight of Salviae Miltiorrhizae;
2% by weight of Codonopsis Radix;
1% by weight of Persicae Semen; and
1% by weight of Ophiopogon japonicus (Linn.f.) Ker-Gawl.

It should be noted that, according to the crude drug dosage recommended by the pharmacopoeia, a dosage of the compound composition prepared herein is 1.8-3.6 g, and an effective dosage of the traditional Chinese medicine composition is determined by subsequent animal experiments.

In addition, the Chinese medicinal materials used in the traditional Chinese medicine extracts disclosed ae scientifically selected, and the Chinese traditional medicine extracts and the index components to be added are determined according to the Chinese classic prescription, network pharmacology and Chinese Pharmacopoeia, specifically:

The medicinal materials added we 4-5 kinds of Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl. The index components are the ginseng total saponin or the ginseng polysaccharide, the beta-Poria polysaccharide or the pachyman, the rehmannioside A, B, C, D or the dihydrocatalpol glycoside or the taurian glycoside, the echinacoside or the Cistanche deserticola Ma polysaccharides or the cistanches glycoside respectively. Tanshinone IIA or cryptotanshinone or salvianolic acid B, Codonnoside or Codonopsis polysaccharide, amygdalin or Persicae Semen polysaccharide, total saponins of Ophiopogon japonicus (Linn.f.)Ker-Gawl. or high isoflavone or ophiopogonis polysaccharide, respectively.

Further, the traditional Chinese medicine extracts ae combined organically according to the compatibility of traditional Chinese medicines rather than simple superimposing of the effect of each Chinese medicine. Effects of the abovementioned Chinese medicine materials we listed as follows:

Panax ginseng C. A. Meyer: ginseng sweet, slightly bitter, slightly warm, belonging to heart, lung, spleen meridians. Greatly invigorating the vital energy, invigorating the spleen and lung, generating saliva, quenching thirst, calming the nerves and increasing intelligence. Ginseng is listed as the top-grade medicine in Shen Nong's Materia Medica, which has the function of “taking more and taking longer without harming the body, lightening the body, benefiting Qi and prolonging life”.

Clinical application of Panax ginseng C. A. Meyer: \textcircled{1} for the critical symptoms of Qi deficiency and weak pulse; \textcircled{2} for the syndromes of shortness of breath, shortness of breath, lazy voice, pulse deficiency and self-sweating; \textcircled{3} for the syndromes of lack of temper, fatigue, lack of food and loose stool; \textcircled{4} for the syndromes of body heat, thirst and thirst caused by heat disease and Qi and fluid injury; \textcircled{5} for the syndromes of palpitation, insomnia, forgetfulness caused by deficiency of Qi and blood.

Poria: sweet, light, flat, belonging to the heart, lung, spleen, kidney meridians. It can promote diuresis and dampness, strengthen spleen and calm heart. Compendium of Materia Medica records that “Poria has a light and penetrating smell: its nature goes up: it produces fluid; it opens up water; it nourishes water and decreases; it benefits urination. So, Zhang Jiegu said that it belongs to Yang, floating and rising, which means its nature; Dongyuan said that it belongs to Yin in Yang, falling and falling, which means its function”.

Rehmannia glutinosa: sweet, cold, belonging to heart, liver and kidney meridian. Nourishing Yin and kidney, nourishing blood and cooling blood. It is quite beneficial for all Yin deficiency, blood deficiency, kidney deficiency. Compendium of Materia Medica records that “filling bone marrow, growing muscle, producing blood essence, invigorating five internal organs and insufficient internal injuries, dredging blood vessels, benefiting ears and eyes, blacking hair, treating five labors and seven injuries in men, cell leakage in women's injuries, irregular menstrual syndrome and fetal diseases”.

Cistanche deserticola Ma: sweet in taste, salty, warm in nature, belonging to kidney and large intestine meridians. Invigorating kidney Yang, invigorating blood essence and moistening intestines. It has the functions of anti-aging, adjusting endocrine, promoting metabolism and strengthening, improving immunity, promoting the synthesis of DNA, and reducing blood pressure. Shennong's materia medica classic records that “it governs five labors and seven injuries, invigorates the middle, eliminates the cold and heat pain in the stem, nourishes the five internal organs, strengthens Yin, benefits essence and Qi, and treats gynecopathy”.

Salviae Miltiorrhizae: bitter, slightly cold, belonging to heart and liver meridians. It promotes blood circulation and remove blood stasis, relieve pain, clear heart and remove trouble, cool blood and eliminate carbuncle. It is used for chest pain, abdominal pain, syndrome accumulation, heat pain, restlessness, irregular menstruation, dysmenorrhea, amenorrhea, sore, swelling and pain. Compendium of Materia Medica records that “activating blood circulation, dredging heart envelope, and treating hernia pain”. “Bielu” records that “nourishing blood, removing chronic diseases of heart and abdomen, accumulating Qi, strengthening waist and spine, blocking feet, removing pathogenic wind and retaining hear, taking for a long time is good for human body”.

Codonopsis Radix: sweet and flat. Invigorating the middle and nourishing Qi, harmonizing the stomach to produce fluid, and relieving cough.

Persicae Semen: bitter, sweet, flat, belonging to heart, liver, large intestine meridians. Promoting blood circulation and removing blood stasis, moistening the intestines and laxative, relieving cough and relieving asthma.

Ophiopogon japonicus (Linn.f.) Ker-Gawl.: sweet, slightly bitter, slightly cold in nature, belonging to the stomach, lungs, and heart meridians. It has the effects of nourishing Yin, nourishing the lung, replenishing the stomach and promoting fluid, clearing the heart and eliminating trouble. Shen Nong's Materia Medica lists Ophiopogon japonicus (Linn.f.) Ker-Gawl. a the top grade for nourishing Yin and moisturizing the lung, saying that it “long service and light body, not old nor hungry”.

Qiongyu ointment is composed of Panax ginseng C. A. Meyer, Rehmannia glutinosa, white Poria and honey. It comes from Hong's Prescription in Song Dynasty. In Yuan Dynasty. Wang Haogu's Yileiyuanrong records that “filling essence and nourishing marrow, making white hair black, rejuvenating and acting like flying feather”. In the Compendium of Materia Medica by Li Shizen of Ming Dynasty, it is said that “if people take it regularly, it can make people happy, develop their intelligence, make their white hair black, and make their teeth grow out again, so as to prolong their life”.

The disclosure is based on modified Qiongyu ointment, strictly following the principle of the compatibility of monarch drugs, ministerial drugs, adjuvant drugs and envoy drugs. Panax ginseng C. A. Meyer is used as a monarch drug to invigorate Qi and body fluid, invigorate spleen and lung, tranquilize mind and intelligence. Cistanche deserticola Ma is used to invigorate kidney Yang, invigorate essence and blood, while Rehmannia glutinosa is used to invigorate kidney Yin, and both of them ae used as minister drugs to invigorate Yin and Yang, and help the monarch drug to consolidate the essence and cultivate Yuan. Poria is used to invigorate the spleen and promote dampness to prevent greasiness and dampness; Salviae Miltiorrhizae is used to promote blood circulation and remove blood stasis, clear the heart and remove annoyance to help the circulation of Qi and blood, so that it is not stagnant; Codonopsis Radix is use to replenish the Qi and promote the stomach; Persicae Semens is used to promote blood circulation and remove blood stasis; Ophiopogon japonicus (Linn.f.) Ker-Gawl. is used to nourish Yin and lungs, clear the heart and eliminate troubles: the above-mentioned flavors we used as adjuvant drugs. The combination of all the drugs can nourish Qi, blood, Yin and Yang, invigorate the kidney, essence and blood, and tranquilize the mind and wisdom.

In particular, the principles of modern medical organization of the present disclosure are as follows.

The research shows that the traditional Chinese medicine is rich in saponins, polysaccharides, flavonoids, polyphenols and other functional ingredients, having various physiological activities such a anti-fatigue, anti-tumor, anti-oxidation, immune regulation, lowering blood sugar and blood lipid, regulating blood pressure, inhibiting bacteria and promoting wound healing. For example, ginsenoside in Panax ginseng C. A. Meyer, Poria polysaccharide in Poria, Rehmannioside, Cistanche deserticola Ma, tanshinone or salvianolic acid B, Codonopsis saponin, etc. have been proved by abundant modern medical research that they have the physiological functions of anti-oxidation, supporting vascular health, immune regulation and so on.

In addition, as a typical marine active substance, chitooligosaccharide has many advantages such as diverse functions, wide sources, strong specificity, little toxic and side effects, and has gradually become a research hotspot in the 21st century. The chitooligosaccharide is widely used in food, cosmetics, biomedicine and other fields. The chitooligosaccharide is the only alkaline oligosaccharide with positive charge in nature. It not only has strong adsorption capacity for all kinds of toxic substances with negative charge in the body, but also has reducibility. Its alkalinity can neutralize acidic substances, improve the constitution, and can specifically combine with cells to improve immunity.

Further, synergism is a common reaction among active substances. As early as the 1980s, it was discovered that there was synergism among different substances, especially in traditional Chinese medicine compounds which is a kind of complex system. In traditional Chinese medicine, the components ae various, and there may be certain synergism among different components. If the proportion is appropriate, the efficiency of the whole food can be improved. In the present disclosure, the synergism of the polysaccharide, the saponin and the flavone of the traditional Chinese medicine extract and the marine oligochitosan chitooligosaccharides is utilized, so that the technical effect of delaying aging is achieved on the basis of modern medicine.

In addition, though the common dosage of each traditional Chinese medicine is known in the prior at, the prescription of the disclosure is made for the target disease by organically combining the above medicines, and its medicinal effect is not equivalent to the simple superposition of the effects of these medicines at a commonly-used amount. Actually, it cannot determine the amount of each medicine in the prescription according to their individual commonly-used amount, and the compounding ratio depends on many factors such as the characteristics of the medicinal materials and the compatibility of monarch drugs, ministerial drugs, adjuvant drugs and envoy drugs, and cannot be determined by experimental means such as comparison method and orthogonal test.

In a second aspect, the disclosure provides a method of preparing the compound composition with anti-oxidation and anti-aging effects, including:

subjecting Panax ginseng C. A. Meyer, Poria, Rehmanniae glutinosa, Cistanche deserticola Ma, Salviae Miltiorrhizae, Codonopsis Radix, Persicae Semen and Ophiopogon Japonicus (Linn.f.) Ker-Gawl. to extraction, purification, concentration and drying to produce Panax ginseng C. A. Meyer extract, Poria extract, Rehmanniae glutinosa extract, Cistanche deserticola Ma extract, Salviae Miltiorrhizae extract, Codonopsis Radix extract, Persicae Semen extract and Ophiopogon japonicus (Linn.f.) Ker-Gawl. extract; and

mixing the Panax ginseng C. A. Meyer extract, the Poria extract, the Rehmanniae glutinosa extract, the Cistanche deserticola Ma extract, the Salviae Miltiorrhizae extract, the Codonopsis Radix extract, the Persicae Semen extract, the Ophiopogon japonicus (Linn.f.) Ker-Gawl. extract and the chitooligosaccharides uniformly to produce the compound composition:

wherein the method specifically comprises:

(1) crushing, sieving, and subjecting Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl. to extraction respectively with an ethanol solution under refluxing and filtration; subjecting a residue to extraction with a solvent according to certain proportions and filtration several times; and combining filtrates to obtain a crude Panax ginseng C. A. Meyer extract, a crude Poria extract, a crude Rehmanniae glutinosa extract, a crude Cistanche deserticola Ma extract, a crude Salviae Miltiorrhizae extract, a crude Codonopsis Radix extract, a crude Persicae Semen extract and a crude Ophiopogon japonicus (Linn.f.) Ker-Gawl. extract, respectively;

(2) subjecting the crude Panax ginseng C. A. Meyer extract, the crude Poria extract, the crude Rehmanniae glutinosa extract, the crude Cistanche deserticola Ma extract, the crude Salvia Miltiorrhizae extract, the crude Codonopsis Radix extract, the crude Persicae Semen extract and the crude Ophiopogon japonicus (Linn.f.) Ker-Gawl. extract to purification and concentration, spray drying or vacuum drying, and sieving to produce the Panax ginseng C. A. Meyer extract, the Poria extract, the Rehmanniae glutinosa extract, the Cistanche deserticola Ma extract, the Salviae Miltiorrhizae extract, the Codonopsis Radix extract, the Persicae Semen extract the Ophiopogon japonicus (Linn.f.) Ker-Gawl, extract; and

(3) mixing the Panax ginseng C. A. Meyer extract the Poria extract, the Rehmanniae glutinosa extract, the Cistanche deserticola Ma extract, the Salviae Miltiorrhizae extract, the Codonopsis Radix extract, the Persicae Semen extract, the Ophiopogon japonicus (Linn.f.) Ker-Gawl, extract and the chitooligosaccharides uniformly in the weight ratio of claim 1 to produce the compound composition with anti-oxidation and anti-aging effects.

Preferably, in step (1), the sieving is performed using a sieve of 20-80 mesh; a volume ratio of Panax ginseng C. A. Meyer to the ethanol solution is 1:(5˜20); a volume ratio of Poria to the ethanol solution is 1:(5˜20); a volume ratio of Rehmanniae glutinosa to the ethanol solution is 1:(5˜20); a volume ratio of Cistanche deserticola Ma to the ethanol solution is 1:(5˜20), a volume ratio of Salviae Miltiorrhizae to the ethanol solution is 1:(5˜20); a volume ratio of Codonopsis Radix to the ethanol solution is 1:(5˜20); a volume ratio of Persicae Semen to the ethanol solution is 1:(5˜20), a volume ratio of Ophiopogon japonicus (Linn.f.) Ker-Gawl, to the ethanol solution is 1:(5˜20); and the extraction of Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f) Ker-Gawl, is performed 1-3 times at 80-100° C. for 1-3 h each time, respectively.

Preferably, in step (2), the spray drying is performed at 180-200° C.; the vacuum drying is performed at 50-100° C.; and the sieving is performed using a sieve of 60-80 mesh.

Preferably, step (2) further includes: before the spray drying or the vacuum drying, introducing β-cyclodextrinto the concentrated Panax ginseng C. A. Meyer filtrate, Poria filtrate, Rehmanniae glutinosa filtrate, Cistanche deserticola Ma filtrate, Salviae Miltiorrhizae filtrate, Codonopsis Radix filtrate, Persicae Semen filtrate and Ophiopogon japonicus (Linn.f) Ker-Gawl, filtrate, respectively, followed by mixing uniformly, wherein the β-cyclodextrinto is 1%-20% by weight of Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl., respectively.

In a third aspect the disclosure further provides an application of the compound composition with anti-oxidation and anti-aging effects in preparation of health food.

Further, the compound composition is used as a raw material of dietary supplement or health food, and a dosage form of the compound composition is a capsule, a granule or a tablet.

Compared with the prior art, the disclosure provides a compound composition with anti-oxidation and anti-aging effects, and preparation method and application thereof, with the following beneficial effects:

1. Compared with traditional Chinese medicine extraction method, the disclosure adopts the preparation process of compound after separate extraction of medicinal materials, which ha more stable extraction quality and higher controllability.

2. The compound composition disclosed and protected by the disclosure has good effects of anti-oxidation, anti-aging and increasing the telomerase content through pharmacodynamic experiments.

3. The compound composition disclosed by the disclosure has no toxic side effects and is easy to be absorbed, is used for delaying aging, repairing DNA damage and anti-oxidation damage, and can also reduce the occurrence risk of geriatric diseases. In addition, the compound composition disclosed can be used as a raw material of dietary supplement or health food, so as to increase the telomerase content and delay aging. It can be seen that the compound composition is suitable for popularization and application.

4. In the present disclosure, multiple granulate and preparation processes are adopted, so that the hygroscopicity of the preparation can be effectively improved, the stability of the finished product can be ensured, and the medicinal effect of the medicine can be enhanced, so as to finally improve the efficacy of the compound composition.

### BRIEF DESCRIPTION OF THE DRAWINGS

In order to explain the embodiments of the present disclosure or the technical solutions in the prior art more clearly, the following drawings that need to be used in the description of the embodiments or the prior art will be briefly introduced. Obviously, the drawings in the following description ae only embodiments of the present disclosure. For those of ordinary skill in the art, other drawings can be obtained based on the drawings disclosed without creative work.

FIG. 1 is the scavenging ability on DPPH free radical of the compound composition of the disclosure.

FIG. 2 is the scavenging ability on hydroxyl radical of the compound composition of the disclosure.

### DETAILED DESCRIPTION OF EMBODIMENTS

Technical solutions of the present disclosure will be clearly and completely described below with reference to the embodiments. Obviously, described below we merely some embodiments of the disclosure, which are not intended to limit the disclosure. Other embodiments made by those skilled in the art without sparing any creative effort should fall within the scope of the disclosure.

The embodiments of the disclosure provide a compound composition, which has no toxic side effect and is easy to be absorbed. The compound composition is used as a raw material of dietary supplement or health food for delaying aging and anti-oxidation. In addition, the compound composition can increase the telomerase content, repair DNA damage and anti-oxidation damage, and can also reduce the occurrence risk of geriatric diseases. The compound composition is suitable for market promotion.

The disclosure will be further described below with reference to the embodiments. It should be understood that these embodiments are merely illustrative of the disclosure, and are not intended to limit the disclosure. Any improvement and modification made by those skilled in the art without departing from the spirit of the disclosure should still fall within the scope of the disclosure.

Hereinafter, the technical solution disclosed will be further described with reference to specific embodiments.

### Embodiment 1

A compound composition with anti-oxidation and anti-aging effects is formulated as follows:

chitooligosaccharides: 50 g, Panax ginseng C. A. Meyer medicinal material: 100 g, Poria medicinal material: 200 g, Rehmannia glutinosa medicinal material: 166 g, Cistanche deserticola Ma medicinal material: 168 g, Salviae Miltiorrhizae medicinal material: 100 g. magnesium stearate: 5 g.

The specific preparation steps of the compound composition are as follows:

(1) The Panax ginseng C. A. Meyer medicinal material is crushed into coarse granules (sieved with a sieve of 20 mesh). The coarse granules were subjected to extraction with a 75% ethanol solution in a volume ratio of 1:8 under refluxing twice for 3 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Panax ginseng C. A. Meyer extract.

(2) The Poria medicinal material is crushed into coarse granules (sieved with a sieve of 80 mesh). The coarse granules were subjected to extraction with water in a volume ratio of 1:10 under refluxing once for 100 min and filtration. Filtrate was concentrated to 1/10 of the original volume, precipitated with 80% ethanol solution for 12 h and filtrated. Then filtrate was added with 10% β-cyclodextrinto and continuously subjected to spray drying at 200° C. The dried product was sieved with a sieve of 80 mesh to produce the Poria extract.

(3) The Rehmanniae glutinosa medicinal material is crushed into coarse granules (sieved with a sieve of 20 mesh). The coarse granules were subjected to extraction with a 60% ethanol solution in a volume ratio of 1:8 under refluxing twice for 1.5 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Rehmanniae glutinosa extract.

(4) The Cistanche deserticola Ma medicinal material is crushed into coarse granules (sieved with a sieve of 20 mesh). The coarse granules were subjected to extraction with a 75% ethanol solution in a volume ratio of 1:8 under refluxing twice for 1 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Cistanche deserticola Ma extract.

(5) The Salviae Miltiorrhizae medicinal material is subjected to extraction with an 80% ethanol solution in a volume ratio of 1:8 under refluxing twice for 1.5 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Salviae Miltiorrhizae extract.

(6) The prepared traditional Chinese medicine extract and chitooligosaccharide were evenly mixed, granulated and filled into compound capsules, which is 100 times of the daily prescription dose.

### Embodiment 2

A compound composition with anti-oxidation and anti-aging effects is formulated as follows:

chitooligosaccharides: 40 g, Panax ginseng C. A. Meyer medicinal material: 72 g. Poria medicinal material: 144 g, Rehmannia glutinosa medicinal material: 120 g, Cistanche deserticola Ma medicinal material: 180 g. Salviae Miltiorrhizae medicinal material: 72 g. magnesium stearate: 3 g.

The specific preparation steps of the compound composition are as follows:

(1) The Panax ginseng C. A. Meyer medicinal material is crushed into coarse granules (sieved with a sieve of 20 mesh). The coarse granules were subjected to extraction with a 75% ethanol solution in a volume ratio of 1:6 under refluxing twice for 3 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Panax ginseng C. A. Meyer extract.

(2) The Poria medicinal material is crushed into coarse granules (sieved with a sieve of 80 mesh). The coarse granules were subjected to extraction with water in a volume ratio of 1:8 under refluxing once for 120 min and filtration. Filtrate was concentrated to 1/10 of the original volume, precipitated with 80% ethanol solution for 12 h and filtrated. Then filtrate was added with 10% β-cyclodextrinto and continuously subjected to spray drying at 200° C. The dried product was sieved with a sieve of 80 mesh to produce the Poria extract.

(3) The Rehmanniae glutinosa medicinal material is crushed into coarse granules (sieved with a sieve of 20 mesh). The coarse granules were subjected to extraction with a 70% ethanol solution in a volume ratio of 1:10 under refluxing twice for 1 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Rehmanniae glutinosa extract.

(4) The Cistanche deserticola Ma medicinal material is crushed into coarse granules (sieved with a sieve of 20 mesh). The coarse granules were subjected to extraction with a 80% ethanol solution in a volume ratio of 1:6 under refluxing twice for 1 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Cistanche deserticola Ma extract.

(5) The Salviae Miltiorrhizae medicinal material is subjected to extraction with an 80% ethanol solution in a volume ratio of 1:8 under refluxing twice for 1.5 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Salviae Miltiorrhizae extract.

(6) The prepared extract and chitooligosaccharide are evenly mixed to obtain the traditional Chinese medicine compound composition, and the granules of the traditional Chinos medicine compound composition are obtained by mixing and granulating, which is 100 times of the daily prescription dose.

### Embodiment 3

A compound composition with anti-oxidation and anti-aging effects is formulated as follows:

chitooligosaccharides: 30 g, Panax ginseng C. A. Meyer medicinal material: 63 g, Poria medicinal material: 103 g, Rehmannia glutinosa medicinal material: 84 g, Cistanche deserticola Ma medicinal material: 157 g, Salviae Miltiorrhizae medicinal material: 63 g. Codonopsis Radix: 52.5 g, Persicae Semen: 52.5 g, magnesium stearate: 5 g.

The specific preparation steps of the compound composition are as follows:

(1) The Panax ginseng C. A. Meyer medicinal material is crushed into coarse granules (sieved with a sieve of 20 mesh). The come granules were subjected to extraction with a 80% ethanol solution in a volume ratio of 1:4 under refluxing twice for 3 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Panax ginseng C. A. Meyer extract.

(2) The Poria medicinal material is crushed into coarse granules (sieved with a sieve of 80 mesh). The coarse granules were subjected to extraction with water in a volume ratio of 1:6 under refluxing once for 1.5 h and filtration. Filtrate was concentrated to 1/10 of the original volume, precipitated with 80% ethanol solution for 12 h and filtrated. Then filtrate was added with 10% β-cyclodextrinto and continuously subjected to spray drying at 200° C. The dried product was sieved with a sieve of 80 mesh to produce the Poria extract.

(3) The Rehmanniae glutinosa medicinal material is crushed into coarse granules (sieved with a sieve of 20 mesh). The coarse granules were subjected to extraction with a 70% ethanol solution in a volume ratio of 1:6 under refluxing twice for 1.5 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Rehmanniae glutinosa extract.

(4) The Cistanche deserticola Ma medicinal material is crushed into coarse granules (sieved with a sieve of 20 mesh). The coarse granules were subjected to extraction with an 80% ethanol solution in a volume ratio of 1:6 under refluxing twice for 1 h each time and filtration. Filtrates were combined, purified, concentrated and added with 10% β-cyclodextrinto. The combined filtrate was continuously subjected to spray drying at 200° C. The dried product was sieved with a sieve of 80 mesh to produce the Cistanche deserticola Ma extract.

(5) The Salviae Miltiorrhizae medicinal material is subjected to extraction with an 80% ethanol solution in a volume ratio of 1:8 under refluxing twice for 1.5 h each time and filtration. Filtrates were combined, purified, concentrated and added with 10% O-cyclodextrinto. The combined filtrate was continuously subjected to spray drying at 200° C. The dried product was sieved with a sieve of 80 mesh to produce the Salviae Miltiorrhizae extract.

(6) The Codonopsis Radix medicinal material is crushed into coarse granules (sieved with a sieve of 20 mesh). The coarse granules were subjected to extraction with a 60% ethanol solution in a volume ratio of 1:8 under refluxing once for 40 min and filtration. Filtrates were purified, concentrated and added with 10% β-cyclodextrinto. The combined filtrate was continuously subjected to spray drying at 200° C. The dried product was sieved with a sieve of 80 mesh to produce the Codonopsis Radix extract.

(7) The Persicae Semen is subjected to extraction with water in a volume ratio of 1:10 under refluxing once for 2 h. The extracted product were purified, concentrated and added with 10% β-cyclodextrinto. The combined product was continuously subjected to spray drying at 200° C. The dried product was sieved with a sieve of 80 mesh to produce the Persicae Semen extract.

(8) The prepared extract and chitooligosaccharide are evenly mixed to obtain the traditional Chinese medicine compound composition, and the granules of the traditional Chinese medicine compound composition are obtained by dry granulation, which is 100 times of the daily prescription dose.

Embodiments provided herein are described in a progressive manner, and each embodiment mainly focuses on the differences from other embodiments. The same or similar parts among various embodiments can be referred to each other.

Further, the following tests were performed to evaluate the efficacy of the compound composition prepared herein.

### (1) Study on Anti-Oxidation Effect of the Disclosure In Vitro

The compound composition preparation and other drug prescriptions prepared in embodiment 1 were used to test the anti-oxidation effect of the compound composition in vitro.

1) Grouping of Test Samples:

Test substance A: 50 g of chitooligosaccharides, 100 g of Panax ginseng C. A. Meyer medicinal material, 200 g of Poria medicinal material, 166 g of Rehmannia glutinosa medicinal material, 168 g of Cistanche deserticola Ma medicinal material, 100 g of Salviae Miltiorrhizae medicinal material, and 5 g of magnesium stearate were prepared into 100 times daily prescription dose according to the method of embodiment 1.

Test substance B: 50 g of chitooligosaccharides, 100 g of Panax ginseng C. A. Meyer medicinal material, 200 g of Poria medicinal material, 166 g of Rehmannia glutinosa medicinal material, 168 g of Cistanche deserticola Ma medicinal material, 33.3 g of β-cyclodextrinto, and 5 g of magnesium stearate were prepared into 100 times daily prescription dose according to the method of embodiment 1.

Test substance C: 50 g of chitooligosaccharides, 100 g of Panax ginseng C. A. Meyer medicinal material, 200 g of Poria medicinal material, 166 g of Rehmannia glutinosa medicinal material, 168 g of Codonopsis Radix, 60 g of Persicae Semen, and 5 g of magnesium stearate were prepared into 100 times daily prescription dose according to the method of embodiment 1.

Test substance D: 50 g of chitooligosaccharides, 100 g of Panax ginseng C. A. Meyer medicinal material, 200 g of Poria medicinal material, 166 g of Rehmannia glutinosa medicinal material, 100 g of Salviae Miltiorrhizae, 168 g of Codonopsis Radix, and 5 g of magnesium stearate were prepared into 100 times daily prescription dose according to the method of embodiment 1.

Test substance E: 50 g of chitooligosaccharides, 100 g of Panax ginseng C. A. Meyer medicinal material, 200 g of Poria medicinal material, 166 g of Rehmannia glutinosa medicinal material, 89.3 g of β-cyclodextrinto, 5 g of magnesium stearate were prepared into 100 times daily prescription dose according to the method of embodiment 1.

Preparation of test samples: the above test substances A-E were diluted with distilled water to four step concentrations of 10, 30, 50 and 100 mg/ml respectively.

2) Test Method

a) DPPH Radical Scavenging Ability

1 ml of sample solution and 1 ml of DPPH-ethanol solution (0.125 mol/L) were mixed in a vortex oscillator. After 30 min of dark reaction at room temperature, the absorbance value was measured at 517 nm. Anhydrous ethanol was used as the blank control, which was recorded as A. All samples were determined for 3 times in parallel, and the average value was taken. DPPH radical scavenging rate was calculated by the following formula:

$$DPPH radical scavenging rate ( \% ) = ( 1 - \frac{{A}_{2} - {A}_{1}}{{A}_{0}} ) \times 100 \%$$

Where: A₁ was the absorbance value of the sample without DPPH solution; A₂ was the absorbance value of DPPH solution and sample.

b) Determination of Hydroxyl Radical Scavenging Rate

1 ml sample solution was added with 1.0 ml FeSO₄ (9 mmol/L), 10 ml salicylic acid ethanol solution (9 mmol/IL) and 1 ml H₂O₂ (9 mmol/L) respectively. After reaction at 37° C. for 1 h, the absorbance of the mixture at 510 nm was determined. Anhydrous ethanol was used as blank control, which was recorded as A₀. All samples were determined for 3 times in parallel. The scavenging ability of hydroxyl radical was calculated by the following formula:

$$Hydroxyl radical scavenging rate ( \% ) = ( 1 - \frac{{A}_{2} - {A}_{1}}{{A}_{0}} ) \times 100 \%$$

Where: A₁ was the absorbance value of distilled water instead of H₂O₂; A₂ was the absorbance value of hydroxyl radical system and sample.

3) Result Analysis

DPPH is a kind of stable free radical with nitrogen as the center. There is a strong absorption peak at 517 nm. The electron or hydrogen atom provided by antioxidant reacts with DPPH, resulting in the decrease of its absorbance value at 517 nm, be change of absorbance value reflects the anti-oxidation ability of antioxidant.

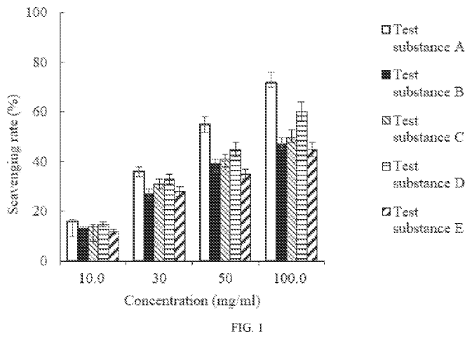

It can be seen from FIG. 1 that the scavenging ability of DPPH radical of each test substance increased with the increase of concentration, and the anti-oxidation ability was as follows: test substance A>test substanceD>test substanceC>test substanceB>test substance E. Therefore, the anti-oxidation effect of test substance A was the best.

Hydroxyl radical is the most active chemical substance in the reactive oxygen system. It can easily pass through the cell membrane and react with biological molecules such as carbohydrate, protein, lipid, nucleic acid and almost any biological component in the cell to induce tissue damage or lead to cell death.

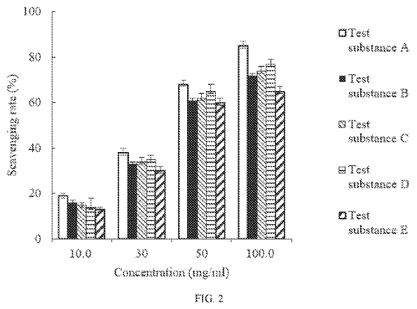

It can be seen from FIG. 2 that with the increase of the mass concentration of test substance, the scavenging ability of each test substance to hydroxyl radical presented a stable growth. The scavenging ability of test substance A was greater than that of other test substances in each mass concentration measurement range. When the mas concentration was 100.0 mg/ml, the scavenging ability of each test substance to hydroxyl radical reached the maximum.

Based on the above results, the disclosure has remarkable anti-oxidation effect. The indexes of test substance A were significantly better than those of the others, that is, the anti-oxidation effect of the compound composition composed of chitooligosaccharide. Panax ginseng C. A. Meyer, Poria, Rehmannia glutinosa, Cistanche deserticola Ma and Salviae Miltiorrhizae was significantly better than that of the compound composition composed of chitooligosaccharide, Panax ginseng C. A. Meyer, Poria, Rehmannia glutinosa and 0-1 kinds or two combinations of Cistanche deserticola Ma, Salviae Miltiorrhizae, Codonopsis Radix, Persicae Semen and Ophiopogon Japonicus (Linn.f.) Ker-Gawl. It shows that the anti-oxidation effect of the disclosure is related to the composition of the specific raw materials of the disclosure. The reasonable proportion of the raw materials of the disclosure, together with the specific raw material extraction method of the disclosure, makes the final prepared compound composition have significant anti-oxidation effect.

### (2) Human Trial

The safety and anti-aging effect of the compound composition capsule prepared in embodiment 1 were tested.

Inclusion criteria: 50-75 years old, normal aging or disease-induced weakness, physical strength and lack of energy;

Exclusion criteria: persons with the following diseases: chronic allergic enteritis or functional diarrhea; autoimmune diseases or immunodeficiency; positive diagnosis of HIV, hepatitis B or hepatitis C; history of severe heart, lung, kidney and liver dysfunction; occurrence of major cardiovascular diseases in the past 6 months; neuropsychological diseases or cognitive impairment diagnosed by medicine; alcoholism and high alcohol intake.

Experimental design and grouping: before and after self-control.

Dosage and method: 3 capsules, 2 times a day, 3.6 g/day for 90 days.

Observation Indicators:

### 1) Security Observation:

\textcircled{1} Blood routine: red blood cell count, average red blood cell volume, red blood cell distribution width, average hemoglobin content, average red blood cell hemoglobin concentration, white blood cell count, monocyte count, monocyte ratio, neutrophil count, lymphocyte ratio, average platelet volume, hematocrit;

\textcircled{2} Urine routine: urine color, urine transparency, urine pH, red blood cells, white blood cells, urine protein, urine ketone body, urine sugar;

\textcircled{3} Blood biochemistry: liver function examination: total bilirubin, total protein, white protein, globulin, albumin/globulin ratio, aspartate aminotransferase, alanine aminotransferase, etc;

\textcircled{4} Renal function: creatinine (CR), blood urea nitrogen (BUN);

\textcircled{5} ECO.

### 2) Efficacy Observation:

\textcircled{1} Blood pressure skin test: skin elasticity, collagen and melanin abnormalities

\textcircled{2} Vascular regulatory factors: nitric oxide (No), vascular endothelial growth factor (VEGF)

\textcircled{3} Superoxide dismutase (SOD) activity and telomerase (TE) content in serum

\textcircled{4} Follow up service scale for experiential personnel:
Aging symptoms: Four symptoms of poor physical strength (fatigue after walking or activities, poor physical strength in daily life, dizziness, poor sleep) were included, and were classified into grade I-IV according to the severity, with statistical integral values.
Grade I (0 point): keeping a certain amount of exercise, energetic; daily energetic; rarely dizzy, only after overwork; falling asleep within 30 minutes, sleep time more than 5 hours, not easy to wake up, not sleepy during the day.
Grade II (1 point): slightly tired after more walking activities (such as walking 2 km); slightly tired, not affecting daily activities; dizziness will occur only once or twice a month, life will not be affected; falling asleep within 30 min. sleep time is less than 5 h, no sleepiness during the day.
Grade III (2 points): feeling between grade II and grade IV; fatigue easily, daily activities affected; dizziness twice a week, aggravating after fatigue, affecting daily life; falling asleep more than 30 minutes, sleeping less than 5 hours, easily awakened, sleepy at a fixed time during the day.
Grade IV (3 points): weak back, dizziness, limb weakness; severe fatigue, severely restricted daily activities; dizziness every day, aggravated after a little activity, seriously affecting daily life; more than 30 minutes to sleep, sleep time less than 5 hours, often wake up in the middle of the night without reason, often sleepy during the day.
Diseases of the elderly and sub-health symptoms: Six items (anemia, anorexia, cold infection, etc., mental unrest/palpitation, chills and pain) were included, and were classified into Grade I-IV according to the severity, with statistical integral values.
Grade I (0 point): No symptoms.
Level II (1 point): Low appetite for food, but normal eating; slight chills; slight anemia, which does not affect life after nutritional supplement, slight pain; only one or two times a month of mental unrest/palpitations; competent for general daily activities, in a little heavy physical activity, heart palpitations aggravated; occasionally cold, easy to fall ill only after season or tired.
Grade III (2 points): difficulty in eating, risk of malnutrition; severe chills, significantly different from normal people; moderate anemia; severe pain in specific parts; restlessness/palpitation twice a week, slightly high activity (walking 2 km), severe palpitation, accompanied by mental distress; cold frequency between U and IV.
Grade IV (3 points): no eating at all, severe malnutrition: extreme fear of cold, serious impact on life; severe anemia, maintaining normal life by drugs; general pain, serious impact on the quality of life; palpitation every day, mental tension, palpitation after slight activity; frequent cold, frequent illness, poor resistance.

TABLE 1
General condition before feeding

| Project | Subjects |
|---|---|
| Number | 30 |
| M/F | 17/13 |
| Age (years) | 50~76 |

TABLE 2
Analysis of blood routine examination results
before and after the test (x ± SD)

| Project | 0 day | 90 days | P value |
|---|---|---|---|
| WBC count (10⁹/L) | 5.96 ± 3.00 | 6.91 ± 3.27 | 0.4729 |
| Monocyte ratio (%) | 9.06 ± 0.87 | 8.13 ± 1.47 | 0.3176 |
| Monocyte count (10⁹/L) | 0.66 ± 0.05 | 0.64 ± 0.17 | 0.7277 |
| Lymphocyte ratio (%) | 40.64 ± 12.07 | 40.55 ± 10.82 | 0.9861 |
| WBC count (10⁹/L) | 5.96 ± 3.00 | 6.91 ± 3.27 | 0.4729 |
| Monocyte ratio (%) | 9.06 ± 0.87 | 8.43 ± 1.47 | 0.3176 |
| Monocyte count (10⁹/L) | 0.66 ± 0.05 | 0.64 ± 0.17 | 0.7277 |
| Lymphocyte ratio (%) | 40.64 ± 12.07 | 40.55 ± 10.82 | 0.9861 |
| Neutrophil ratio ratio (%) | 57.99 ± 11.29 | 57.20 ± 13.98 | 0.8960 |
| Neutrophil count (10⁹/L) | 2.01 ± 1.33 | 2.20 ± 1.39 | 0.8123 |
| Red blood cell distribution | 11.06 ± 1.31 | 12.28 ± 1.41 | 0.1141 |
| width (%) | | | |
| Mean corpuscular volume (fL) | 90.98 ± 7.99 | 91.57 ± 8.38 | 0.8335 |
| Mean platelet volume (fL) | 9.97 ± 2.19 | 10.38 ± 1.66 | 0.6770 |
| Hematocrit (%) | 0.30 ± 0.07 | 0.31 ± 0.08 | 0.8535 |
| Mean hemoglobin (pg) | 31.95 ± 1.61 | 31.93 ± 1.82 | 0.9387 |
| Mean hemoglobin | 359.81 ± 1.63 | 353.81 ± 6.93 | 0.2187 |
| concentration (g/L) | | | |

TABLE 3
Analysis of liver and kidney function and blood biochemical indexes
before and after the test (x ± SD)

| Project | 0 day | 90 days | P value |
|---|---|---|---|
| Albumin (g/L) | 36.94 ± 0.87 | 38.11 ± 1.19 | 0.3713 |
| White ball ratio | 1.12 ± 0.07 | 1.65 ± 0.30 | 0.1395 |
| Triglyceride (mmol/L) | 2.24 ± 0.77 | 2.10 ± 0.66 | 0.6378 |
| High density lipoprotein | 1.43 ± 0.47 | 1.46 ± 0.42 | 0.8386 |
| (mmol/L) | | | |
| Low density lipoprotein | 3.72 ± 0.71 | 3.86 ± 0.77 | 0.6312 |
| (mmol/L) | 6.29 ± 1.09 | 6.19 ± 1.13 | 0.8341 |
| Total cholesrol (mmol/L) | | | |
| Fasting blood glucose (mmol/L) | 8.00 ± 1.57 | 7.47 ± 1.43 | 0.3695 |

TABLE 4
Analysis of heart rate before and after the test (x ± SD)

| Project | Number | 0 day | 90 days | P value |
|---|---|---|---|---|
| Heart rate | 30 | 69.60 ± 11.59 | 73.23 ± 12.61 | 0.2579 |
| (BPM) | | | | |

There was no obvious abnormality in chest X-ray, electrocardiogram, abdominal ultrasound and urine routine examination before and 30 days after the test. During the test period the subjects had no adverse reactions or allergic reactions.

### Test 1

TABLE 5
Effective rate of the compound composition in improving aging symptoms,
diseases of the elderly and sub-health symptoms

| | Effective rate/% | | |
|---|---|---|---|
| | | Improving diseases | |
| | | of the elderly and | |
| | Improving aging | sub-health | |
| Time | symptoms | symptoms | Total |
| 30 days | 82.76% | 73.08% | 96.67% |
| 60 days | 86.67% | 73.33% | 100% |
| 90 days | 90.00% | 86.67% | 100% |

It can be seen from the above Table 5 that the compound composition can improve the aging symptoms, diseases of the elderly and sub-health symptoms, and the effective rate increases gradually with the extension of taking time; after taking the compound composition for 60 days, the total effective rate of improving the aging symptoms can reach 100%, indicating that the compound composition of the disclosure has extremely significant effect on improving aging.

### Test 2

TABLE 6
Effective rate of the compound composition in improving
skin condition of the elderly

| | Effective rate/% | | |
|---|---|---|---|
| | improving skin | improving | improving |
| | elasticity | collagen | melanin |
| Time | abnormality | abnormality | abnormality |
| 30 days | 27.59% | 20.69% | 31.03% |
| 60 days | 27.83% | 21.05% | 30.95% |
| 90 days | 28.61% | 22.17% | 32.04% |

The above table 6 shows that the compound composition can improve the skin elasticity, collagen and melanin abnormalities of the elderly to a certain extent, and the effective rate of improvement increases with the extension of taking time.

### Test 3

TABLE 7
Effect of the compound composition on improving
anti-aging enzyme content in the elderly people

| | content/% | |
|---|---|---|
| | Superoxide | |
| Time | dismutase | Telomerase |
| 0 day | 16.90 ± 9.23 | 1.17 ± 1.05 |
| 30 days | 22.11 ± 9.45* | 1.59 ± 0.88* |
| 60 days | 26.57 ± 10.91** | 1.64 ± 0.64* |
| 90 days | 29.08 ± 6.31** | 1.72 ± 0.31** |

Note:
compared with 0 day,
*P <0.05; compared with 0 day,
**P <0.01

In Table 7, the anti-aging enzyme content in the elderly people is detected, and it is found that the longer the compound composition was taken, the higher the contents of the superoxide dismutase and the telomerase am. The compound composition of the disclosure can effectively improve the anti-aging enzyme content in the elderly people.

TABLE 8
Effective rate of the compound composition in improving
anti-aging enzyme in elderly people

| | Effective rate/% | |
|---|---|---|
| | Improving superoxide | Improving |
| Time | dismutase | Telomerase |
| 30 days | 86.67% | 79.31% |
| 60 days | 88.30% | 82.16% |
| 90 days | 90.08% | 86.07% |

It can be seen from the data in above Table 8 that the effective rate of the compound composition to improve the anti-aging enzyme in elderly people reached the highest on the 90th day of the test, and the effective rates of superoxide dismutase and telomerase were 90.08% and 86.07% respectively, indicating that the compound composition of the disclosure will gradually improve the effective rate of the anti-aging enzyme in elderly people with the extension of the taking time, which has a relatively good antioxidant effect.

### Test 4

TABLE 9
Effect of the compound composition on improving the
content of vascular regulatory factors in elderly people

| | Content/% | |
|---|---|---|
| | | Vascular endothelial |
| Time | Nitric oxide (NO) | growth factor (VEGF) |
| 0 day | 21.80 ± 29.34 | 26.85 ± 27.27 |
| 30 days | 47.17 ± 26.20** | 47.87 ± 29.84** |
| 60 days | 50.27 ± 21.98** | 54.08 ± 20.48** |
| 90 days | 53.08 ± 25.32** | 57.29 ± 21.03** |

Note:
compared with day 0 **P <0.01

It can be seen from the above table 9 that the contents of nitric oxide and vascular endothelial growth factor in the body of the subject, taking the compound composition by the disclosed method showed an upward trend, and increased with the extension of taking time, indicating that the compound composition has the effect of increasing the content of vascular regulatory factors in the body.

TABLE 10
Effective rate of the compound composition in improving
vascular regulatory factors in elderly people

| | Effective rate/% | | |
|---|---|---|---|
| | Improving | Improving vascular | |
| | Nitric | endothelial gowth | |
| Time | oxide (NO) | factor (VEGF) | Total |
| 30 days | 93.10% | 93,10% | 96.55% |
| 60 days | 95.24% | 94.82% | 98.99% |
| 90 days | 97.10% | 96.04% | 99.20% |

It can be seen from the data in Table 10 above that the effective rate of compound composition in improving vascular regulatory factors in elderly people increased with the extension of taking time. On the 90th day, the total effective rate was 99.2% and the effective rates of NO and VEGF were 97.10% and 96.04% respectively. It is suggested that the compound composition has the functions of dilating blood vessels, promoting blood vessel regeneration and protecting cardio cerebral vessels.

Based on the analysis of the above results, it can be seen that the compound composition has a significant anti-aging effect and effectively improves the aging signs of the elderly, including aging, diseases and sub-health symptoms. The effective rate is high, with an overall effective rate of 100%. It can effectively improve the skin condition of the elderly, and enhance the contents of anti-aging enzyme and vascular regulatory factors in the elderly.

The above results show that the compound composition disclosed by the disclosure has anti-oxidation and anti-aging effects. The compound composition has no toxic side effects and is easy to be absorbed. It can be used to delay aging, repair DNA damage and anti-oxidation damage, and reduce the risk of geriatric diseases, which is suitable for market promotion and application.

Described above are merely illustrative of the disclosure to enable those skilled in the art to implement or use the disclosure, and ae not intended to limit the disclosure. It should be understood that any modification, replacement and change made by those skilled in the art without departing from the spirit of the disclosure should fall within the scope of the disclosure.

1. A compound composition with anti-oxidation and anti-aging effects comprised of traditional Chinese medicine extracts and chitooligosaccharides according to a mass ratio of (3-8): 1; wherein the traditional Chinese medicine extracts are prepared by extracting the following Chinese medicinal materials respectively and compounding, according to a mass percentage:
7%-15% of Panax ginseng C. A. Meyer;
14%˜35% of Poria;
14%˜35% of Rehmanniae glutinosa;
14%˜35% of Cistanche deserticola Ma;
7%˜15% of Salviae Miltiorrhizae;
0%˜10% of Codonopsis Radix,
0%˜10% of Persicae Semen; and
0%˜10% of Ophiopogon japonicus (Linn.f.) Ker-Gawl.

2. The compound composition of claim 1, wherein the traditional Chinese medicine extracts are prepared by extracting the following Chinese medicinal materials respectively and compounding, according to a mass percentage:
12%-15% of Panax ginseng C. A. Meyer,
25%˜30% of Poria;
20%˜25% of Rehmanniae glutinosa;
20%˜25% of Cistanche deserticola Ma;
12%˜15% of Salviae Miltiorrhizae;
2%˜3% of Codonopsis Radix,
1%˜2% of Persicae Semen; and
1%˜2% of Ophiopogon japonicus (Linn.f.) Ker-Gawl.

3. The compound composition of claim 1, wherein the traditional Chinese medicine extracts ae prepared by extracting the following Chinese medicinal materials respectively and compounding, according to a mass percentage:
13% of Panax ginseng C. A. Meyer;
26% of Poria;
22% of Rehmanniae glutinosa;
22% of Cistanche deserticola Ma;
13% of Salviae Miltiorrhizae;
2% of Codonopsis Radix;
1% of Persicae Semen; and
1% of Ophiopogon japonicus (Linn.f.) Ker-Gawl.

4. A method of preparing the compound composition of claim 1, comprising:
subjecting Panax ginseng C. A. Myer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl, to extraction, purification, concentration and drying to produce Panax ginseng C. A. Meyer extract, Poria extract, Rehmanniae glutinosa extract, Cistanche deserticola Ma extract, Salviae Miltiorrhizae extract, Codonopsis Radix extract, Persicae Semen extract and Ophiopogon japonicus (Linn.f.) Ker-Gawl, extract; and
mixing the Panax ginseng C. A. Myer extract, the Poria extract, the Rehmanniae glutinosa extract, the Cistanche deserticola Ma extract, the Salviae Miltiorrhizae extract, the Codonopsis Radix extract, the Persicae Semen extract, the Ophiopogon japonicus (Linn.f.) Ker-Gawl, extract and the chitooligosaccharides uniformly to produce the compound composition;
wherein the method specifically comprises:
(1) crushing, sieving, and subjecting Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl, to extraction respectively with an ethanol solution under refluxing and filtration; subjecting a residue to extraction with a solvent according to certain proportions and filtration several times; and combining filtrates to obtain a crude Panax ginseng C. A. Meyer extract, a crude Poria extract, a crude Rehmanniae glutinosa extract, a crude Cistanche deserticola Ma extract, a crude Salviae Miltiorrhizae extract, a crude Codonopsis Radix extract, a crude Persicae Semen extract and a crude Ophiopogon japonicus (Linn.f.) Ker-Gawl, extract, respectively;
(2) subjecting the crude Panax ginseng C. A. Meyer extract, the crude Poria extract, the crude Rehmanniae glutinosa extract, the crude Cistanche deserticola Ma extract, the crude Salviae Miltiorrhizae extract, the crude Codonopsis Radix extract, the crude Persicae Semen extract and the crude Ophiopogon japonicus (Linn.f.) Ker-Gawl, extract to purification and concentration, spray drying or vacuum drying, and sieving to produce the Panax ginseng C. A. Meyer extract, the Poria extract, the Rehmanniae glutinosa extract, the Cistanche deserticola Ma extract, the Salviae Miltiorrhizae extract, the Codonopsis Radix extract, the Persicae Semen extract, the Ophiopogon japonicus (Linn.f.) Ker-Gawl extract; and
(3) mixing the Panax ginseng C. A. Meyer extract, the Poria extract, the Rehmanniae glutinosa extract, the Cistanche deserticola Ma extract, the Salviae Miltiorrhizae extract, the Codonopsis Radix extract, the Persicae Semen extract, the Ophiopogon japonicus (Linn.f.) Ker-Gawl, extract and the chitooligosaccharides uniformly in the weight ratio of claim 1 to produce the compound composition with anti-oxidation and anti-aging effects.

5. The method of preparing the compound composition of claim 4, wherein in the step (1), the sieving is performed using a sieve of 20-80 mesh; a volume ratio of Panax ginseng C. A. Meyer to the ethanol solution is 1:(5˜20); a volume ratio of Poria to the ethanol solution is 1:(5˜20); a volume ratio of Rehmanniae glutinosa to the ethanol solution is 1:(5˜20) a volume ratio of Cistanche deserticola Ma to the ethanol solution is 1:(5˜20); a volume ratio of Salviae Miltiorrhizae to the ethanol solution is 1:(5˜20); a volume ratio of Codonopsis Radix to the ethanol solution is 1:(5˜20); a volume ratio of Persicae Semen to the ethanol solution is 1:(5˜20); a volume ratio of Ophiopogon japonicus (Linn.f.) Ker-Gawl to the ethanol solution is 1:(5˜20); and the extraction of Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl, is performed 1-3 times at 80-100° C. for 1-3 h each time, respectively.

6. The method of preparing the compound composition of claim 4, wherein in the step (2), the spray drying is performed under 180-200° C. the vacuum drying is performed under 50-100° C.; and the sieving is performed using a sieve of 60-80 mesh.

7. The method of preparing the compound composition of claim 6, wherein the step (2) further comprises:
before the spray drying or the vacuum drying, introducing β-cyclodextrinto the concentrated Panax ginseng C. A. Meyer filtrate, Poria filtrate, Rehmanniae glutinosa filtrate. Cistanche deserticola Ma filtrate, Salviae Miltiorrhizae filtrate, Codonopsis Radix filtrate, Persicae Semen filtrate and Ophiopogon japonicus (Linn.f.) Ker-Gawl, filtrate, respectively, followed by mixing uniformly;
wherein the β-cyclodextrinto is 1%-20V by weight of Panax ginseng C. A. Meyer, Poria, Rehmanniae glutinosa, Cistanche deserticola Ma, Salviae Miltiorrhizae, Codonopsis Radix, Persicae Semen and Ophiopogon japonicus (Linn.f.) Ker-Gawl, respectively.

8. An application of the compound composition of claim 1 in a preparation of health food.

9. The application of claim 8, wherein the compound composition is used as a raw material of dietary supplement or health food, and a dosage form of the compound composition is a capsule, a granule or a tablet.
